(19) United States
(12) Patent Application Publication
(10) Pub. No.: US 2023/0293137 A1
(43) Pub. Date: Sep. 21, 2023

(54) METHOD AND DEVICES FOR IMPROVED PHONOCARDIOGRAPHY AND METHODS FOR ENHANCED DETECTION AND DIAGNOSIS OF DISEASE

(71) Applicant: DIGIBEAT HEALTH MONITORING SYSTEMS, LLC, GAINESVILLE, FL (US)

(72) Inventors: KARL PAUL DOCKENDORF, BLUE BELL, PA (US); WINSTON THOMAS RICHARDS, GAINESVILLE, FL (US); CLIFFORD DARYL STEELE, GAINESVILLE, FL (US)

(21) Appl. No.: 18/122,178

(22) Filed: Mar. 16, 2023

Related U.S. Application Data
(60) Provisional application No. 63/320,240, filed on Mar. 16, 2022.
(60) Provisional application No. 63/345,052, filed on May 24, 2022.

(51) Int. Cl. A61B 7/00; G06T 7/73; H04R 1/46; G06F 3/16; A61B 5/00

(52) U.S. Cl. CPC A61B 7/003; G06T 7/73; H04R 1/46; G06F 3/167; G06F 3/162; A61B 5/7435; G06T 2207/30196; G06T 2207/10016; G06T 2200/24; A61B 2562/0261

(57) ABSTRACT

A digital stethoscope with additional sensors for measuring orientation, positional changes, infrared radiation, strain, and electrophysiology is disclosed. Enhancements to AI driven system(s) and methods as a result of the additional sensors are disclosed. Methods and device implementations enabled by this feature set include providing instruction to a user for improved use of the digital stethoscope in obtaining sound from the body of a patient, automatic exam segmentation, automatic subject orientation capture, and motion capture not possible with a traditional stethoscope transducer alone. A further disclosure of a method for managing heart failure is described.

### CROSS-REFERENCE TO RELATED APPLICATIONS

This application claims the benefit of U.S. Provisional Ser. Nos. 63/320,240, filed Mar. 16, 2022 and 63/345,052, filed May 24, 2022, the disclosures of which are hereby incorporated by reference in their entirety, including all figures, tables and amino acid or nucleic acid sequences.

### BACKGROUND OF INVENTION

Well-being, disease, and changes thereof are important aspects of health to monitor on a routine basis. Well-being is often tracked over time and repeated measurements taken to observe trend changes. Monitoring of disease often requires specialized penetrating sensing modalities requiring great expense to own and operate. Simplified and noninvasive sensing and monitoring technologies are often preferred to enable easier access to patient condition(s). One such sensing and monitoring technology that is difficult for human operators to utilize effectively to obtain information is a phonocardiography or listening (auscultation) through a stethoscope. The stethoscope has long been used by physicians as a sensing modality to diagnose disease and guide the evaluation of physical exam findings. Advancements in Artificial Intelligence (AI) in combination with auscultation devices have made it possible to resolve some conditions more effectively than human skills alone. Moreover, body sounds come from physical phenomena, the frequency components, of which, can be outside the range of human hearing (e.g., heart sound frequencies can extend below 20 Hz). Furthermore, body sounds are known to change with patient orientation. For example, auscultation of the heart of a patient in a reclined versus upright position can affect the sound because vertical changes in the pumping of blood volume changes the load on the heart. The same is true for auscultation sites at the carotid or femoral arteries. In addition, orientation of a patient also impacts the weight “above” organs such as when listening to organs in the abdomen. Knowing these differences is important to understanding the context of what is being heard and thus what physiological processes can be occurring. It would be beneficial if accumulated patients' data pertaining to multiple conditions and body orientations could be gathered through self-directed exams for analysis of sounds from a patient' body to automate diagnosis or to estimate health condition.

### BRIEF SUMMARY

Embodiments of the subject invention pertain to devices and methods for providing guidance to a user for obtaining sounds from the body of a patient, collecting the sounds obtained, and analyzing the sounds to determine a physical condition of the patient. The devices and methods can include devices for acquiring and displaying phonocardiogram (PCG) data and methods for processing PCG data. More specifically, application of advanced processing methods, including artificial intelligence (AI), can enable display and analysis of sensor data obtained from a patient. Disclosed are all-in-one, networked, and cloud-based devices and a plurality of algorithms that make it possible to estimate the health of a patient and diagnose disease states.

Further enhanced, digital stethoscope can be used for motion sensing and obtaining other biometric data in addition to PCG data, which can be utilized together to segment exam sequences, resolve stethoscope positioning, and serve as additional inputs for AI processing. In one embodiment, infrared (IR) sensing is combined with digital stethoscope position mapping to resolve core body temperatures. Electrodes can be utilized for measuring impedance, conductance, and voltages further aiding the diagnostic perspective and features for AI processing. One or more devices can also be provided to allow for viewing, listening, haptics, or otherwise perceiving the information. The acquired information can then be processed by the AI algorithms to determine, for example, risks or likelihood of disease states, tracking and predicting disease trajectory (or state through time), cohorting subjects, estimating physiological parameters, estimating data quality, and guiding positioning of the digital stethoscope.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description are specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1 illustrates an embodiment of the system architecture including a digital stethoscope end, a display, and processing resources, according to the subject invention.

FIG. 2 illustrates multiple views of one embodiment of a digital stethoscope, according to the subject invention.

FIG. 3 shows a diagram of potential auscultation sites.

FIG. 4 shows a diagram of one embodiment of the processing flow for artificial intelligence, regression, or classification algorithms, which can be used with embodiments of the digital stethoscope, according to the subject invention.

FIG. 5 shows an example of a visualization of the attribution by inspection of a diagnostic artificial intelligence (AI) and display overlaid with the digitized stethoscope signal and frequency components.

FIG. 6 shows a diagram of one embodiment of the processing flow for disease-specific, attribution-enhanced, sound amplification.

FIG. 7 shows a diagram of one embodiment of the processing flow for artificial intelligence driven, digital stethoscope repositioning and guidance, with sufficiency determination and user interaction.

FIG. 8 shows an example of visualization of repositioning guidance.

FIG. 9 is a depiction of multiple body and stethoscope orientations and states.

FIG. 10 is a flowchart depiction of an embodiment of a process for digital stethoscope location estimation through integration of sound and accelerations, according to the subject invention.

FIG. 11 is a flowchart depiction of a method for detecting an accelerometer and data acquisition and segmentation.

FIG. 12 is a flowchart depiction of an algorithm for heat mapping and core body temperature estimation.

FIG. 13 is a flowchart depiction of a heart failure treatment methodology.

FIG. 14 is a screen shot from a mobile application that can be utilized to guide a patient in obtaining biometric data with a digital stethoscope.

FIG. 15 is a screen shot from the mobile application in FIG. 14 showing the biometric data that was obtained with the digital stethoscope.

### DETAILED DISCLOSURE

The subject invention pertains to a digital stethoscope and related devices for implementing one or more algorithms and display schemes that aid in collecting and interpreting patient data acquired with the digital stethoscope. The term “stethoscope” refers to any device capable of being utilized as a directional vibration transducer. A vibration transducer refers to a device for detecting or sensing vibrations within or outside the range of human hearing, including, but not limited to, infrasound, ultrasound, and audible sound. In one embodiment, directionality is provided by a stethoscope bell, which guides sound (or vibrations) detected by the bell to a digitization device to be converted to an electrical signal. This can include directing sounds within or outside the range of human hearing, as well as sounds generated by the use of reflecting lasers or ultrasound off (or through) a surface to measure the vibrations, using a solid bell digital stethoscope rather than an air gap, or other contact or non-contact vibration measurements. Embodiments include a digital stethoscope and support systems for algorithms, display, and user interaction.

The term “patient” as used herein, describes an animal, including mammals, to which the devices and methods of the present invention can be applied and that can benefit from such application. This can include mammalian species such as, but not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and, any wild or non-domesticated animal.

The term “user” as used herein refers to the operator or the person manipulating the digital stethoscope device on the body of a patent. The user can be the patient, another person manipulating the stethoscope device on the body of a patient, or some combination thereof.

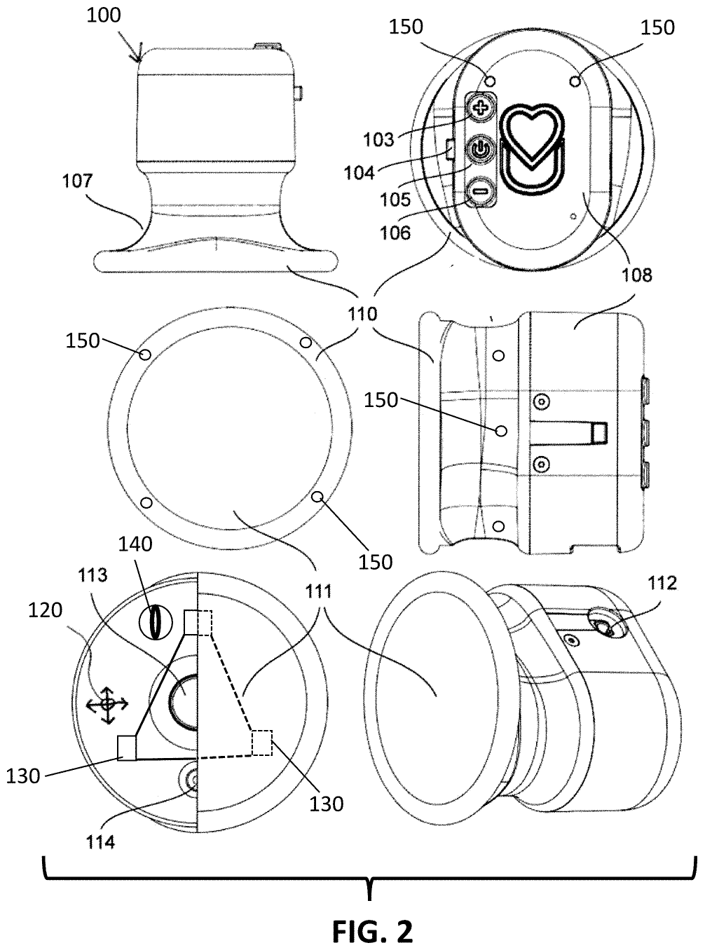

One embodiment of a digital stethoscope 100 has a membrane 111 backed internally by a bell 110 and terminated with a transducer 113 as shown in FIG. 2. A housing 108 can contain a battery and electronic assemblies to condition and digitize a transduced signal. The digitized signal can be transformed, filtered, or otherwise processed to prepare it for listening, viewing, haptic signally, or other methods of perception, or for further computation. The embodiment of the digital stethoscope shown in FIG. 2 incorporates an output port 112 designed for wired listening devices. The digital stethoscope can further incorporate components for wireless transmission of the digitized signal from to any compatible device. The digital stethoscope can have a power button 105, such as, for example, a “+” button 103, and a “−” button 106, providing for user control of the device's power state and outputs. Additional buttons 104 can be added to immediately signal actions without using an application interface. Such shortcuts can trigger functions, such as, for example, recording an audio stream. In another example, it can allow playback and/or processing to a local or remote device, to trigger the end of a continuously overwriting recording cycle. This function can be used to capture a rare event in hindsight, or to switch between processing or filtering modalities (for example, to listen to a known frequency band of a problem or subcomponent). This embodiment or similar directional vibration transduction capabilities can be employed to acquire data in a system with processing and/or visual feedback.

Furthermore, the digital stethoscope 100 can include any of a variety of sensors for detecting and transmitting the physical status of a patent or other patient data. In one embodiment, shown in FIG. 2, an infrared (IR) sensor 114 is located behind the membrane 111. The infrared sensor can be used to measure and approximate the temperature of the patient. Measurement can be adjusted for an estimated core temperature. The adjustment made can be based on typical adjustments and/or based on additional information from processing the digital stethoscope signal (such as, for example, based on the predicted sensing location). IR sensing through the membrane can ensure the IR sensor is directed at the patient, when the membrane is placed against the patient. The measurements obtained with the IR sensor can reflect the patient temperature and inhibit interference from the user.

The digital stethoscope 100 can further include one or more accelerometers 120 and/or gyroscopes 140, which are sensors to detect and measure motion of the digital stethoscope 100. Motion can be integrated over time to estimate changes in the position of the device. In one embodiment, the digital stethoscope has one or more accelerometers to measure directional accelerations. In a further embodiment, the digital stethoscope has one or more gyroscopes to sense and/or measure rotational acceleration. In a still further embodiment, the digital stethoscope has one or more compasses to determine a world aligned absolute orientation without differentiating. The additional information from these positional devices can be utilized by the algorithms of the system to enhance performance and results. Understanding the orientation of the digital stethoscope can aid in determining the orientation or posture of the patient. Orientation or posture of the patient can be important to the interpretation of the sounds obtained by the digital stethoscope.

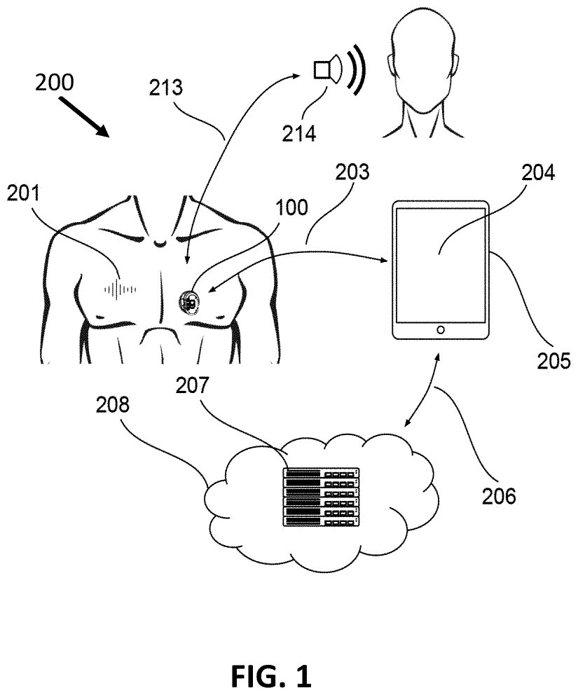

This system 200 of sensing, listening, processing, and/or visualization, as shown, for example, in FIG. 1 can include individual components arranged in various configurations, or the components can be incorporated into an all-in-one device. Some embodiments include: a) sensing, processing and display in one device b) one or more sensors in a separate device, which can be used to contact the patient or make nearer measurements and another device can obtain standoff measurements, such as, for example, image data or provide the display, c) one where significant processing tasks are off-loaded to a supplemental compute resource, d) one where outputs are provided in a data format directly, rather than incorporating a display, for record integration or display through a separate system that can aggregate other information, and e) one where outputs are auditory to directly listen to sounds detected (with or without processing) or analysis results are converted to a synthesized voice. These differentiations could be combined in numerous combinations to produce obvious variations.

In one embodiment of the system 200, as shown in FIG. 1, incorporates the digital stethoscope 200 to sense, detect, or otherwise acquire internal body sounds 201. The sounds can be transmitted 213 for audio or visual review, or some combination thereof. By way of a non-limiting example, the sounds can be transmitted to a hands-free device 214, such as wireless earphones, to a mobile device 205, such as, for example, a mobile phone with a display for visual review, or any other user interface device. In a particular embodiment, the mobile device can have a display screen 204 on which a minimally processed waveform and frequency distribution 206 can be viewed and can be transmitted 206 for further analysis on supplemental compute resources 207. The supplemental compute resources can be maintained on networked machines or can be provisioned in the cloud 208. The analysis results can be transmitted 206 to the mobile device 205 for viewing on the display screen 204. A further embodiment supports the connection of the wireless earphones directly to the mobile device. In this embodiment, the signal to the mobile device can be forwarded to the earphones. This can facilitate replay of recorded and/or processed signals for listening without changing the connection pattern. Selections can be made on the mobile device that influence the components of the signal, which can focus the user on specific sounds or patterns. The digital stethoscope can be used in numerous applications where isolating and/or amplifying internal sounds is desired.

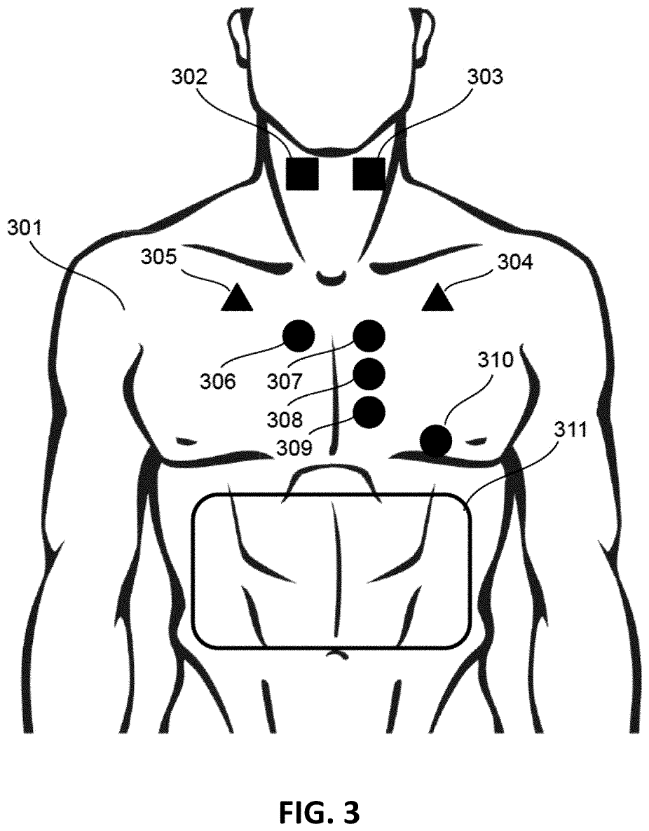

Embodiments of the subject invention are particularly advantageous in the fields of medical and veterinary medicine, but can be beneficial in other fields where auditory signals are indicative of various conditions, such as automotive diagnostics. In the medical context, many auscultation sites can be utilized throughout the human body 301, examples of which are shown in FIG. 3. The system and the digital stethoscope device used therewith are not limited to use with a single organ or system; rather, body sounds 201 can be detected nearly everywhere on a human body 301, though, many optimal locations can be utilized for detecting specific sounds. For example, sounds generated by the heart could be obtained by making observations near the organ, preferably, with minimal rigid material between the organ and the detection device. Examples are seen in the circles in FIG. 3 (306, 307, 308, 309, 310). Alternatively, if breathing or, specifically, the bronchi of the lungs were of interest, observations can be made at sites shown with the triangles 304, 305 in FIG. 3. In another alternative, sounds of digestion can be observed inside the region labeled 311, and blood flow in the carotid artery can be observed at sites 302 and 303, as shown in FIG. 3. Analogous optimal sites exist on most mammals and other animals which are exploited in veterinary medicine and livestock breeding and analysis. In much the same way, automotive diagnostics have optimal observation points depending on the component of interest and thus the potential sources of issues.

In the context of (AI) models for decoding system state, be it valvular disease in a heart or engine valves not sealing in a car, having the greatest signal to noise can be optimal. This is not, however, a requirement of successful data regression to create a performant algorithm or model. In many cases, designing for suboptimal input can be important, such as if it is possible the device will be operated by an unskilled user. Moreover, suboptimality detection can be the goal of AI to guide a user to make better, if not optimal, observations such that the user, device, and algorithm acting in concert results in maximal system performance. As a specific example, the aortic valve is usually best heard in the right, second intercostal space 306, as shown in FIG. 3. It can be difficult for a user to position the digital stethoscope 200 in this optimal position, therefore the ability to utilize suboptimal sounds that can be obtained and provide relevant feedback to the user can be important.

The aforementioned use examples can be enhanced through data-driven application specific algorithms utilized by supplemental compute resources 207. Examples include artificial intelligence (AI), neural networks, regression, Markov models, Gaussian mixture models, support vector machines, random forest, and other machine learning (ML) algorithms and methods for the diagnosis, guidance, detection, screening, monitoring, risk analysis, and prognostication, which we henceforth refer to as artificial intelligence or machine learning models. In the medical context, these can be applied to any number of ailments or current health conditions of the body.

In the medical context, these machine learning models can be applied to any of a variety of ailments or health conditions of the body. In general, two kinds of data are collected to train machine learning models: cross-sectional and longitudinal. In the cross-sectional case, one set of inputs can be mapped to another set of outputs. In the context of disease, for example, a single, brief, 10 second, PCG input can be modeled to predict the heart failure severity or current physiological parameter, such as regurgitation. In a cross-sectional dataset that utilizes similar PCG input, a machine learning model can be regressed or trained to understand the relationship between the PCG signal (or features) and estimate a patient's current regurgitation causes or severity. In a longitudinal dataset, multiple time points are used, potentially collecting changes in PCG signals, severities of events, abnormal events, etc. Then portions of the sequence of information can be used to predict current, or more importantly, subsequent, events or outputs, such as, for example, the severity of heart failure. This provides a tool, wherein an algorithm can predict the time to clinical worsening or the clinical state at some point in the future.

One embodiment of the subject invention is a device implementing a heart age metric. Using heart sounds and age information of patients, a model can be trained to predict the patient's age from the heart sound. Subsequent sounds are estimated to mostly likely be from a patient of the determined specific age. This model relies on a wear concept of the heart as it ages and changes in vasculature, since the resistance of the circulatory system often affects how the heart works. This has health monitoring applications where individuals are interested in monitoring their heart due to hereditary conditions, for positive feedback related to cardiovascular exercise, or any number of other situations. Furthermore, the result can be simple for users to understand: naturally occurring changes are okay and everyone ages, yet there is common perception that health declines with age after 18 years old. Thus, being accelerated in age for adults has negative implications, and the inverse is also true. Age associated data pertaining to physiological changes are more readily acquired and can provide a wealth of information for training models, as opposed to models designed for specific disease pathologies.

This methodology can be extended to other body systems and organs, including one that uses a lung age metric. These datasets can be augmented with other (potentially user entered) data such as smoking history, which can greatly affect many organs. Addition of the information in a machine learning framework, allows for separate understanding of cohorts of healthy patients versus cohorts of patients with suboptimal health or conditions that can skew the input distribution toward pathologic expressions. Moreover, mortality information can be applied to normalize the concept of “age” given the life expectancies of each cohort. Historical and current behavioral data can be one form of additional input and training data, however, additional information comes in the form of other sensor measurements, too. For example, blood pressure can be separately measured and input into the system for more accurate diagnosis.

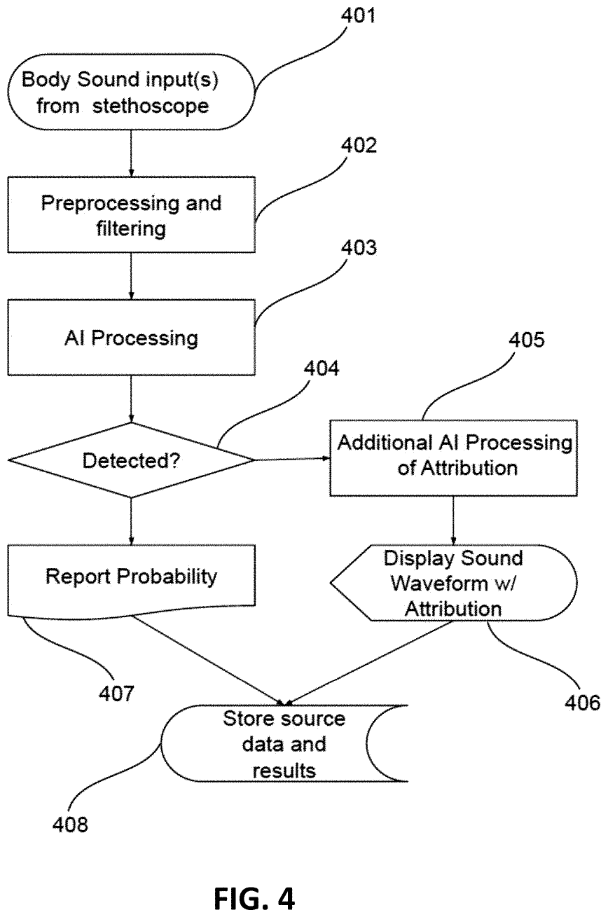

Furthermore, data can also be applicable directly to pathology detection, severity determination, and risk stratification. For example, patient record information, such as Coronary Artery Disease (CAD) state, can be regressed against the sounds sampled from the heart. Turbulence in arteries, changes in body sounds 201 due to wall motion abnormalities, and alterations in the pumping cycle duration and conduction paths along the heart wall can all affect the body sounds that are observed. The learned model can then be applied to undiagnosed individuals (FIG. 4) to determine a CAD state.

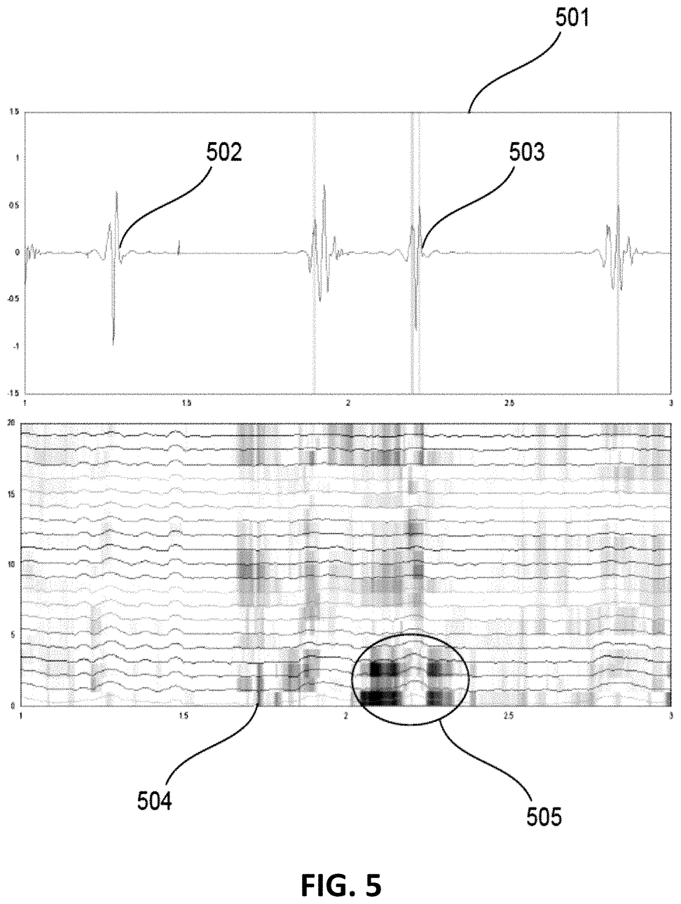

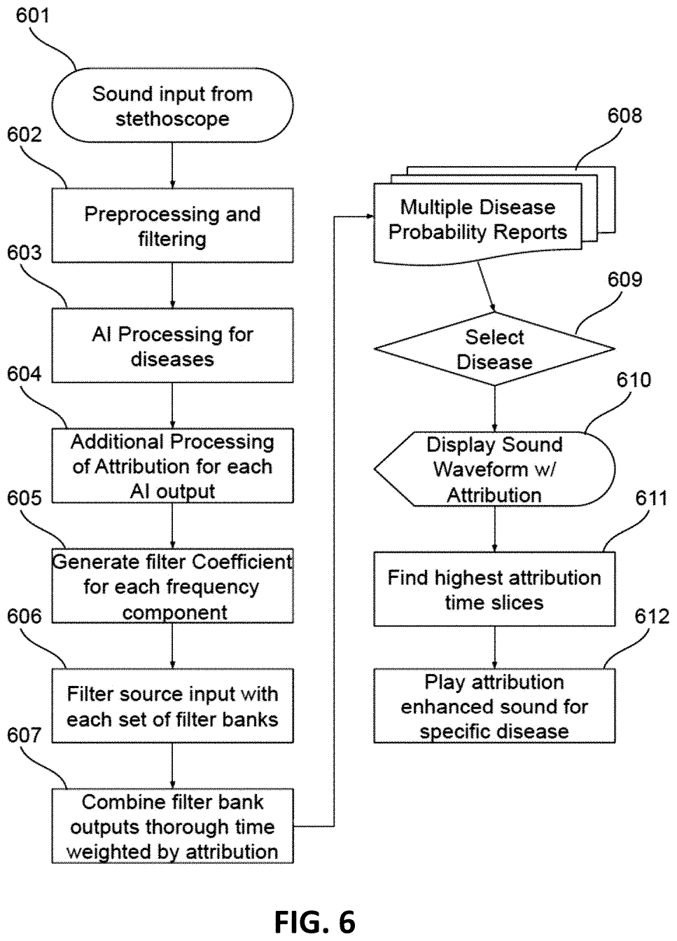

In one embodiment, shown in the flow chart in FIG. 4, a method for acquiring body sounds 201 can be initiated by placing a digital stethoscope on a patient's chest so the body sounds from the heart are transduced and transferred to the point of processing 401. In a further step, the body sounds can be preprocessed and filtered 402 to, for example, enhance the signal-to-noise ratio, alter the sampling rate, change scales, normalize, and/or otherwise prepare the body sounds for processing by the AI. The AI can proceed to process 403 the body sound data to determine a likelihood of one or more pathologies or regress physiologic parameters. For each pathology, a threshold can be used to determine whether further processing is needed depending on the likelihood, probability, or severity 404 of the attribute detected 505, as shown in FIG. 5, associated and the relevant pathology. Given a high enough result, additional processing 405 can be performed with the AI model to determine the contributing portions or “attributes” of the input signal. This “attribution process” enables several features. First, localizing the attribution in the signal focuses deeper analysis or review processes. Second, it minimizes the required storage 408 for preserving evidence of pathology affliction, since only the portion containing the attribute of interest needs to be saved. Third, it enables downstream refiltering of the input signal to highlight or amplify the key signal in a context (FIG. 6). However, in this context, after attribution processing, the waveform can be displayed on screen with the key timeframes highlighted. One embodiment of this highlighting is a display 406, such as shown, for example, in FIG. 5. The user can then evaluate the signal to determine those points in a key timeframe that demonstrate the best examples of the pathology and can listen to those points in the signal. In one embodiment, if the probability does not exceed the threshold for detection, no attribution is computed and instead the low probability can be reported to the user 407. The processing results can be stored 408 for later review, archival purposes, or to prevent reprocessing of the data.

The embodiment above illustrates the algorithms that can be utilized by the supplemental compute resource in the system 200. Furthermore, in a patient context, the method shown in FIG. 4 can be used to map, for example: auscultation site determination, stenosis estimation, valve condition estimation, regurgitation estimation, disease detection, or work/stress estimation of heart or lungs. The system additionally applies to non-human animals and mechanical systems. For example, phenotyping of livestock presents tremendous value for breeding. Where phenotyping is a process that includes determining various parameters of the vitality of animals, particularly the heart.

Datasets that comprise heart sound recordings from a group of patients at one or more points in time can be utilized to predict patient mortality 5 (five) (or N) years later. With a dataset of one sample per individual and mortality information providing a precise time frame after the recording, it is possible to build a model similar to the usage of the data case above that is regressed against future data providing a predictive output. However, a more flexible model that can be employed that uses multiple samples of the intended inputs and outputs such as, for example, a dataset containing multiple heart sound recordings obtained from annual health exams and the corresponding diagnosis dates. Using that dataset, a model can be regressed to map sequences of heart sounds and a query time point to an individual diagnosis state. Then, a whole heart sound history can be input to the algorithm and then queried for the time frame of interest. This enables a user to use the stethoscope system to track progress toward their future selves or understanding their rate of deterioration or the disease trajectory in general.

In one embodiment, this trajectory through time is processed and displayed for one subject relative to a population. This can be performed in many ways, for example using statistical measures of the signal, through latent AI model features, or by dimensionally reducing input, intermediate, or output values of the model. Plotting of such data can give a visual context that simplifies understanding of the patient condition and probable future. Over time, the user will be able to view the effect of any changes to the subject by interventions. Moreover, it can visually confirm that interventions are having a desired effect.

In another embodiment, an AI is trained to predict future interventions and optimum timing therefore. When the subject is monitored using the device, the processing results can show when future interventions may be needed. For example, stenosis in a catheter that increases over time can be monitored for progression and when intervention will be necessary. The digital stethoscope 100 can be used to listen to the flow inside the catheter. The sounds (potentially due to the turbulence of the fluid) are processed by AI to infer the current state of the catheter and, using the history of measurements and/or typical progressions, predict when the catheter's stenosis will exceed prescribed conditions. Then, necessary interventions can be scheduled in advance to remedy the upcoming conditions. There are possible applications for nearly every condition, including but not limited to valve surgery, mechanical valve replacements, fistulas, and peripheral artery disease.

AI decision attribution can build trust with the user 405, 406, result in effective use of time, enable the user to make accuracy decisions using prior knowledge, and automatically annotate data for future AI training. These aspects can also build tremendous value from the perspective of tool utility and compounding data richness. Attribution is a developing interest in the increasingly complex field of AI models. Here, AI attribution methodologies are additionally applied for the development of a novel disease process isolation and noise reduction algorithm and workflow (FIG. 6). While illustrated with attribution methods, these novel workflows can also be built upon back calculations of attention, disambiguated latent variables, secondary models specifically trained for explanatory AI, or other forward calculations of input data importance.

In one embodiment, after acquisition 401, 601 and processing of the data 403, 405, 601-604 as shown in FIGS. 4 and 6, the user is presented with the detected pathologies or conditions. For example, the user can be presented with the current estimated severity of a patient's coronary artery disease. For each condition under inspection, the original and/or transformed representations of the input signal are shown with regions of interest highlighted for further scrutiny by a user or expert (FIG. 5). The visualization can consist of the original source signal 501, a smoothed signal, frequency components of the signal 504, subsections or excerpts from the signal 505, features, latent variables, transformed or filtered signals, or any combination thereof. The signal can also be broken into discreet timeframes 506. The workflow's highlighting of the best examples 503 and the lack of some segments 502 in demonstrating the pathology both improve the user's confidence in the technique, especially for intermittent signals embedded in a larger or longer signal and improves usage time. In other words, this both confirms a user's inability to detect the markers of a pathology in an expected component of the signal for some instances and focuses the user to the easiest to confirm instances reducing the burden for user to AI consensus. As shown in FIG. 5, a heat map overlaid on the signal 501 can provide visual cues for quickly evaluating the accuracy of the data. Automatic segmentation of the periodic signals used in conjunction with the aggregated attribution information provides a discrete list of rankable instances. This can be preferred for lengthy signals and affords the ability to, per period, assign confidence values or probabilities.

Beyond demonstration of the signal of interest (shown via attribution), an enhanced filtering technique is disclosed to amplify the components of the signal relevant to diagnostic outputs. In one embodiment, shown in FIG. 6, sound is input 601, processed for disease processes of interest 602, 603, and source data components can be attributed to the results in step 604. Then, time-varying, source-component dependent weights are generated for use as filter coefficients 605 for reconstruction or reprocessing of the source signal with respect to the disease process of interest. As an example, the original source signal can be filtered by frequency bands or components (similar to a spectrogram or into mel frequency based sub-bands 504). The AI's predicted likelihood of disease (step 603) and the integrated gradients method of attribution (step 505, in FIG. 5 and step 604 in FIG. 6) of this likelihood to the source sub-bands are used to weight the filter bank components like a common audio equalizer being continuously adjusted to amplify sounds related to the disease process. After normalizing the attribution values and scaling the sub-band signals, then each clip is reconstituted via summation, as shown in step 607, of the element-wise product, shown in step 606, of the time-varying sub-band importance signal and the source sub-band components that results in a time-varying signal that can be specific to evidence for each diagnosis made. This resulting isolated evidence signal can be human expert understandable especially in context of the original signal.

The processed results are reported to the user or reviewer, as shown in step 608. They can be presented with each estimated likelihood in an easy-to-understand format. Elements which mark elevated likelihood are selectable for further inspection, as shown in step 609. Once selected additional information is provided with regard to the disease or condition of interest and a playable waveform presented, where regions are annotated or highlighted to direct attention to regions of the signal, as shown at step 610. Regions of the signal, a list of subsamples, or a scrubbable cursor preplaced at the best example, step 611, allow the user to select priority listening regions to playback the sounds, as shown at step 612.

During playback of a processed signal, the user can be presented with a slider or pre-configured options for noise reduction, signal isolation, and/or signal amplification levels. Mapping the AI rationale back to components of the original signal and providing an interface to the user to magnify the basis for determination or suppress the original signal's distracting components, creates learning opportunities, trust, and enables the user to decide whether to rely on the AI's results. These culminate in improved combined user plus AI system performance and relationship with the goal of achieving super-human performance through contextualization of results and sample collection. This is particularly the case with an expert user, however, it may not be the case with untrained users which rely solely on AI guidance.

The AI guidance system enables untrained and trained users to collect specific or multi-site data (i.e., from multiple specific recording sites). This system consists of several logical components: a position and quality inference component, a reference and historic data baseline component with a stopping method, and a positioning and repositioning display.

With respect to the position and quality inference component, one embodiment is a machine learning algorithm that simultaneously computes both measures, trained using ground truth position information and expert scored quality. The advantage of simultaneously estimating position and quality can be that they are linked due to a prior for the desired position of the sensor measurement. Expert rated quality can be based on the ability to identify sound subcomponents expected in a specific position. For example, the aortic valve sounds when auscultated at the aortic site, if instead the mitral site were auscultated other sounds may be clear but the aortic valve may not be. Trial data can be expert re-rated in the context of several desired sounds or position contexts. The expected clarity can be, but is not limited to, being dependent only on position, several other factors are at play. For example, the strength of the heart, the amount and kind of tissue between the sound source and the surface of the body, external material between the skin/stethoscope interface (i.e., clothes, hair, oils, undergarments, etc.). These increase sound attenuation while others increase ambient noise. Ambient noise can come from speech (internal or external to the subject), background nuisances, or purposeful sounds. Quality can then be a measure of this overall clarity of target sources.

The reference and historic data baseline component determines the expected achievable quality. This expected achievable standard can be specific to each user or subject as the system learns specific information about the user, such as, for example, BMI, specificity with which they relocalize (how good they are at targeting and retargeting a spot with guidance), expected achievable background noise, and expected sound levels. Embodiments of one component of the subject invention can determine user feedback like positioning movements, alterations in the environmental noise, alterations in the device contact, and that user has achieved the AI's expectations and can complete the acquisition procedure. This component can use the results from the position and quality estimator such as the position, the quality, foreground and background sound levels, detectability of characteristic components, and others. In addition, the statistical information of these outputs from prior acquisition attempts by this user on this subject, statistical information of these outputs from cohort users or subjects, and statistical information of these outputs without prior information can also be employed in determination of the stopping condition.

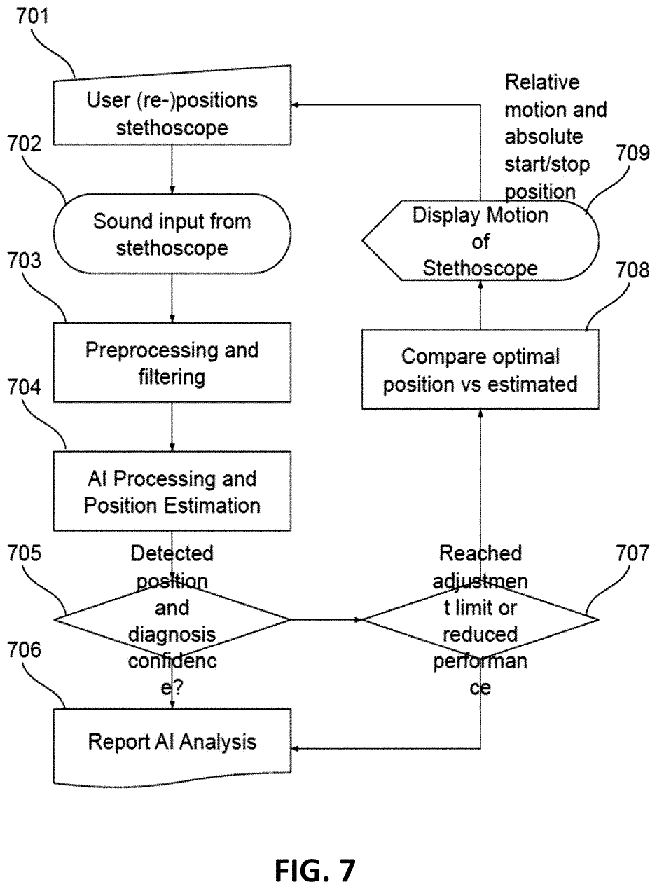

With reference to FIG. 7, it can be seen that following the acquisition 702 and processing of the data steps 703 and 704, one of several display results occur which can include: a) acquisition and analysis complete with display of results, step 706, b) successful acquisition and progression to recording at a new site 701, c) additional user information input requested step 707, or d) suggestions for re-acquisition and new guidance 708. The output given can be determined by the detection and/or estimation algorithm(s) that is(are) desired to be processed, the stopping determination from the reference and baseline component, and divergences from expected results determined by the reference and baseline unit. An acquisition completion state can trigger processing of downstream AI algorithms and notifications for the user. This can occur when all of the necessary data has been acquired. When downstream processing completes, further notifications can be provided to the user. Users are notified of successful acquisition at one site, which can be followed by a request to reposition 709 for further data acquisition for downstream algorithms. Further data acquisition can be preplanned, determined as a result of processing data collected so far, or from user input. Additional user input can be requested to confirm or input suspected reasons for divergences from the expected reference data (overweight, hairy, clothed, uncontrollable ambient noise, etc.). Those inputs can trigger acceptance of previously collected data, changes in the determined cohorts, and/or shifts in the guidance presented to the user. Suggestions for re-acquiring at the same data source can occur due to needed and expected higher quality data. Typically, this occurs along with guidance for re-positioning, alterations to the environment, and/or changes in behavior (stillness, quietness, hold breath, etc.).

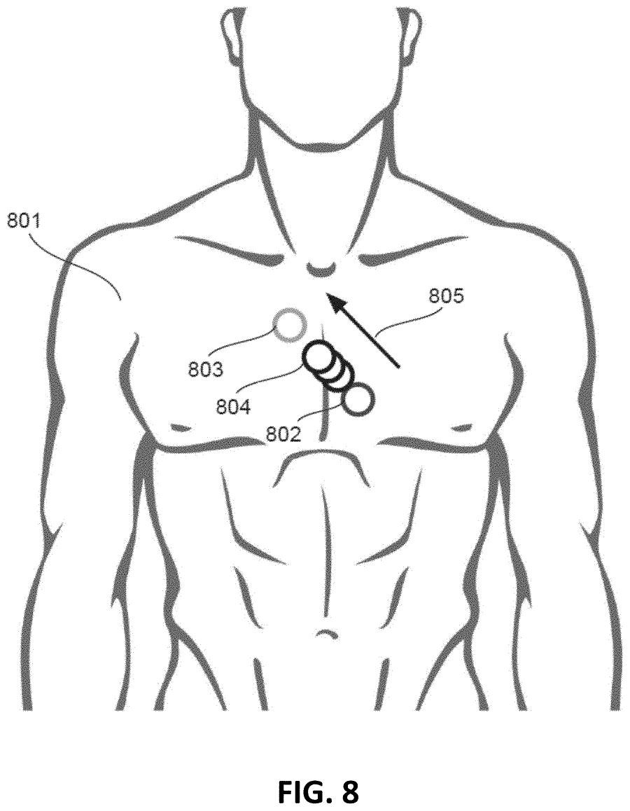

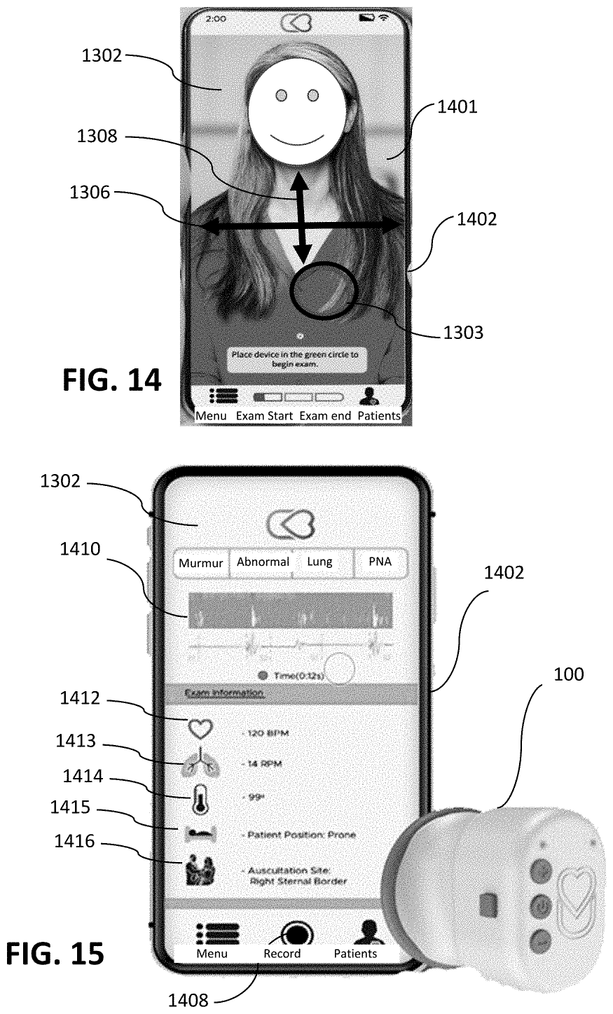

The nature and extent of repositioning guidance (for resampling or new samples) can be important in enabling the user to acquire the intended data source. In FIG. 8, the starting position 802 is shown as being displayed on a homunculus 801 with the desired end position 803. Guidance highlights the movement of the device using, for example, direct queues 805 giving the orientation of the motion independent of the magnitude of the motion. For animated guidance 804, in one embodiment, a fading history, shown by lighter or different colored indicators, of prior positions can be more easily understood—starting near to the current or starting position and moving or relocating towards the desired position. Animated repositioning guidance (as shown in the example in FIG. 8) provides a natural method for a user to understand the guidance, but this does not preclude the use of textual or auditory directions. While the animation of the action is not strictly required for understanding of the guidance, animation can accentuate, thereby easing directive understanding, the motion required while the temporal end points of visual guidance demonstrate the accurate position and placement of the device. It can also be helpful if the user is presented with guidance relative to the patient of the body. In a specific embodiment, the system 200 comprises one or more devices for obtaining an image and/or a video of the patient body, such as shown in FIG. 14. In one embodiment, an image or video of the body of the patient can be obtained prior to or at the time of the examination. The image or video of the body of the patient can be utilized on the user interface device 205, such as, for example, a mobile phone, and utilized to provide the guidance to the user. In specific embodiment, the user interface device is utilized to obtain one or more of an image or video of the body of the patient or some relevant portion thereof.

In one embodiment, a face-on view can be shown for guidance directions. In a further embodiment, additional viewpoint specific views can also be given to aid the understanding of the guidance more rapidly. For example, if the user is the patient, then an additional egocentric view can be presented. In the case of a heart exam, an apparent over the shoulder view or a view looking down at oneself can increase the patient's left/right motion determination, as such view does not need to be reversed from the face-on view. Users that are the patient can occur in many scenarios. For example, when the user is a remote patient or an isolated patient. If the user is not the patient, then the face-on view is representative of the exact experience of the motion required. Supplementary, side, or back facing views would be appropriate when a user is auscultating the back of the patient or when using alternative stances. The direct queues 805 may not be suitable for user viewport-based views, as the user's view can be directly in line with the three-dimensional ray formed by the guidance. However, three dimensionally rendered animated guidance 804 showing a device relocating by moving toward or away from the viewport can be understandable from depth queues.

The captured view of the user can further be used to determine an estimate of the positioning of the stethoscope and the pose of the patient. The relative information can be used to determine current or desired chest placement. Another method is to directly estimate the relative chest positioning using a deep neural network. These methods can be implemented with feature extraction and matching algorithms, or through deep neural networks trained to make direct estimates. For example, marker tracking on a device has pre-implemented solutions like ‘apriltags,’ as well as off-the-shelf pose estimation models like ‘movenet’ for human bodies. Furthermore, image-based implementation of the stethoscope position estimation can be used to enhance position estimation from sounds of interest, as well as on their own. In general, the estimation of the position of the user enables drawing on screen the desired position 1303 in an augmented view of the user on the screen 1402.

Pose estimation of the user can be used as a basis for user feedback or initialization. For example, the aforementioned pose estimation model finds nose, eye, ear, shoulder, elbow, hand, waist, knee, and foot of the user in the frame. By simple comparison of position values in the frame, one can determine if the user is: facing the camera, vertical with respect to the camera, a good distance away from the camera for analysis, and positioned well in the frame. With satisfactory conditions, it is possible to define a relative coordinate system using the shoulders to estimate a position where various heart sounds can be sensed. In one embodiment, a shoulder to shoulder line segment 1306 and it's perpendicular bisector line segment 1308 can be used to define a relative coordinate system that scales with the width of the user's shoulders in the image frame. In relative terms, predefined coordinates for auscultation sites 306-309 are scaled. Then a marker 1303 is drawn on the image to display to the user an initial position to begin an exam. Feature tracking can be used to observe the placement of the stethoscope, which can be limited to the region of the hand (from the pose estimation) to speed processing. Even if the stethoscope is not directly visible (for example, if obscured by the hand) the pose of the hand can be used estimate positioning or provide additional evidence for positioning if used in conjunction with sound features.

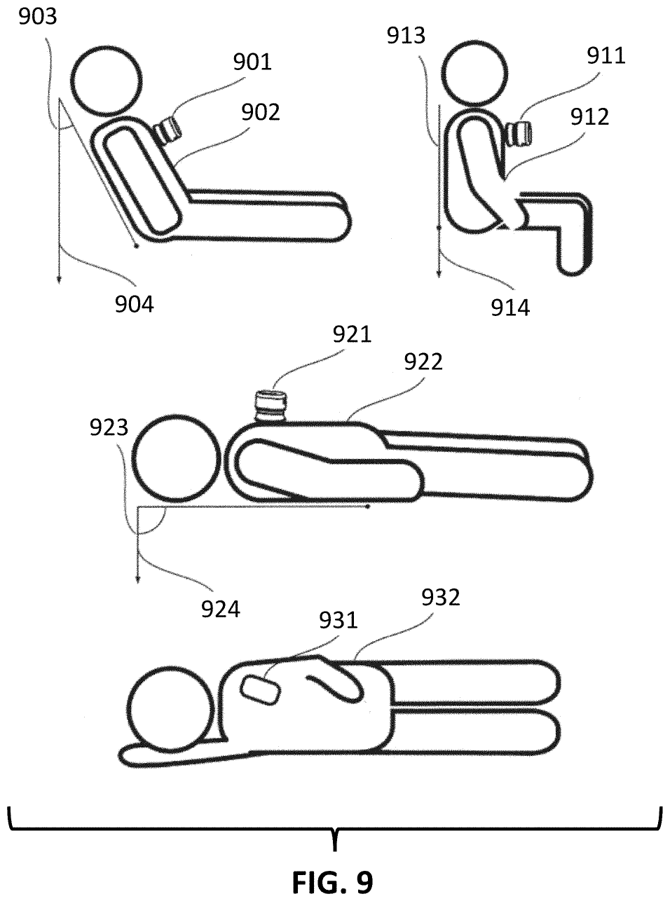

Certain embodiments of the subject invention pertain to a method of utilizing a stethoscope 100 having an internal accelerometer 120 to resolve potential physical positioning or orientations of a patient, as shown, for example, in FIG. 9. Further embodiments utilize the acquired sound data information from the stethoscope to improve analysis of the sound data. In one embodiment, a gravity vector (904, 914, 924) can be read from the accelerometer indicating “down.” From the down vector, a tilt angle (903, 913, 923) of the device can be calculated and, thus, the tilt angle of a recording site. Further, an inferred recording sight location on the body (901, 911, 921, 931) as determined by AI from processing the sounds, at least one orienting angle of the monitored body region can be determined. The estimated orientation of the patient (902, 912, 922, 932) can be stored for display when replay or the stored sounds are requested. This can add context to archival usage of the stethoscope recordings, for example when comparing a history of heart sounds through time. Furthermore, the added orientation data can be directly integrated into AI algorithms in concert with sound data to increase the accuracy of the sound analysis and orientation estimation. The unified algorithm or AI architecture can create internal representations that mix sound and accelerometer features leading to robust representations that resolve, across the spectrum of possible orientations and the spectrum of possible sites, the likeliest combination.

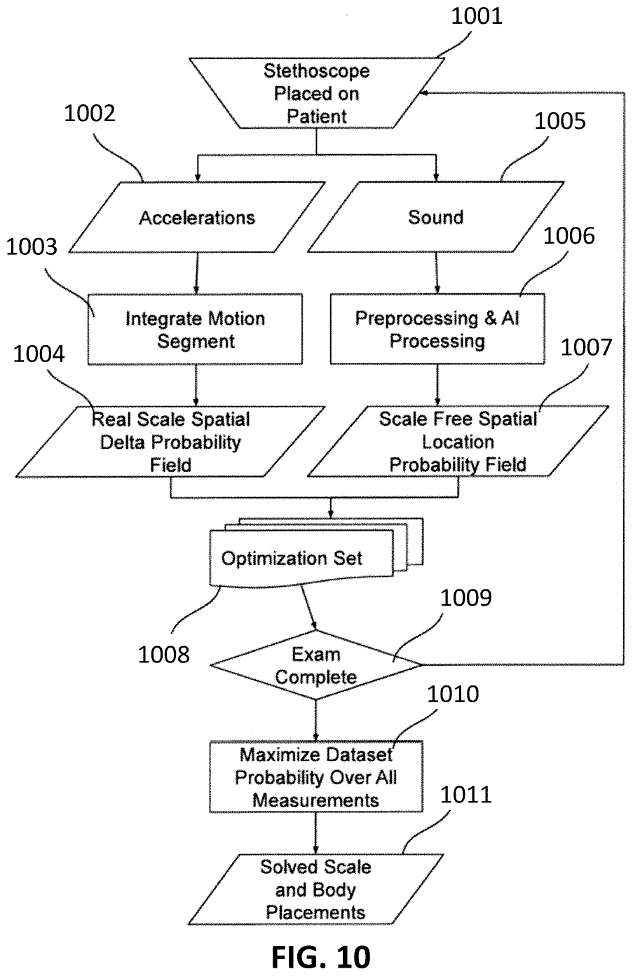

In a further embodiment, the invention is extended to multi-site measurements and analysis. The possible orientations of the body regions can be further constrained by repeating the procedure at a site that is orthogonal or on a different geometric plane, and/or by additional AI processing that detects the most likely positions from sound. The accelerometer 120 of the digital stethoscope 100 can provide both the orientation at each site, and the differences in position and/or orientation between the two or more sites. An embodiment is shown in a flowchart in FIG. 10, where the stethoscope is placed at a target location on a patient body 1001 to acquire accelerations (1002) and sounds (1005). The accelerations can be integrated 1003 into a real-world scale, spatial change probability field 1004 considering the characteristics of the sensor noise. The sound can be preprocessed and analyzed by an AI 1006 to generate a scale-free location probability field 1007 over a homunculus 801, such as shown in FIG. 8. The scale-free location probability field can be shown on the image or video of the patient body. These fields can further be added to an optimization set of data 1008 and the procedure repeated for additional recording sites if desired until the exam is complete 1009. This larger system of measurements forms the basis of a unified optimization problem whereby the device maximizes the dataset probability over all of the measurements obtained 1010. Finally, the AI processes all data together to solve for the best solution for subject condition, subject orientation, subject scale, and the exam sequencing 1011. In one embodiment, this includes the maximization over the world-scale and scale-free probability fields such that the highest probability solution is found.

Moreover, when monitoring multiple sites such that a previous position estimate is available, accelerometer data can be integrated to calculate an initial estimate for the position of subsequent auscultation sites. For example, if aortic valve sounds are determined to be present at one site, that can indicate proximity to the location of the aortic valve. Then, given accelerometer readings indicating motion of the distance and direction that separates the aortic valve and tricuspid valve, there are strong initial conditions for assuming that sounds being heard likely contain the tricuspid valve sounds. This can be further extended by using an estimated body size (through height and weight measurements, for example), to increase the accuracy or normalize across body sizes. Further, this combination can be used as a verification method of non-expert users in acquiring the correct data (or from the correct site). One embodiment of verification is a system in which the user is directed and places the digital stethoscope 100 on the patient. The system can estimate the position of the stethoscope based on sensor measurements. Then, the user can relocate the stethoscope to a new specific site and given real time feedback as to the correct positioning by using the expected change in the body sound source.

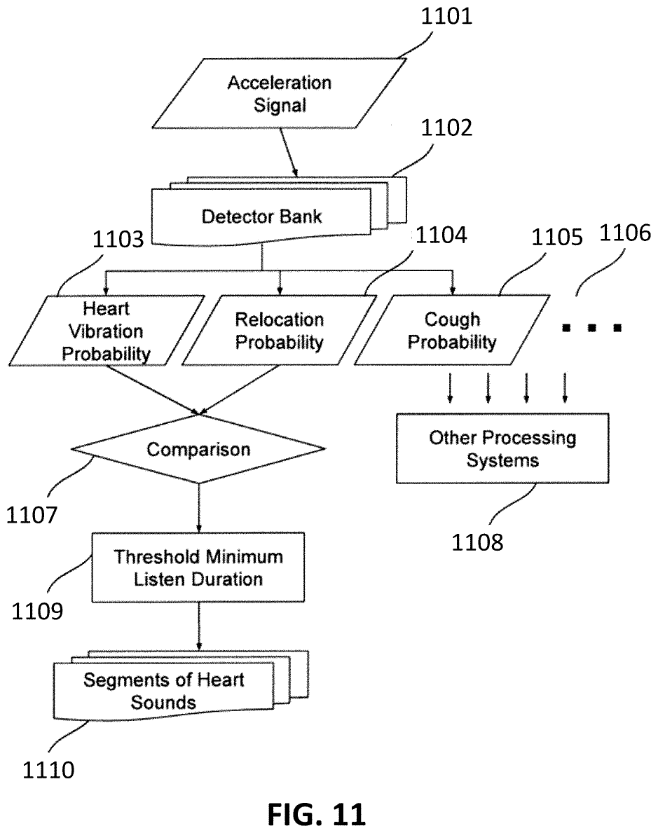

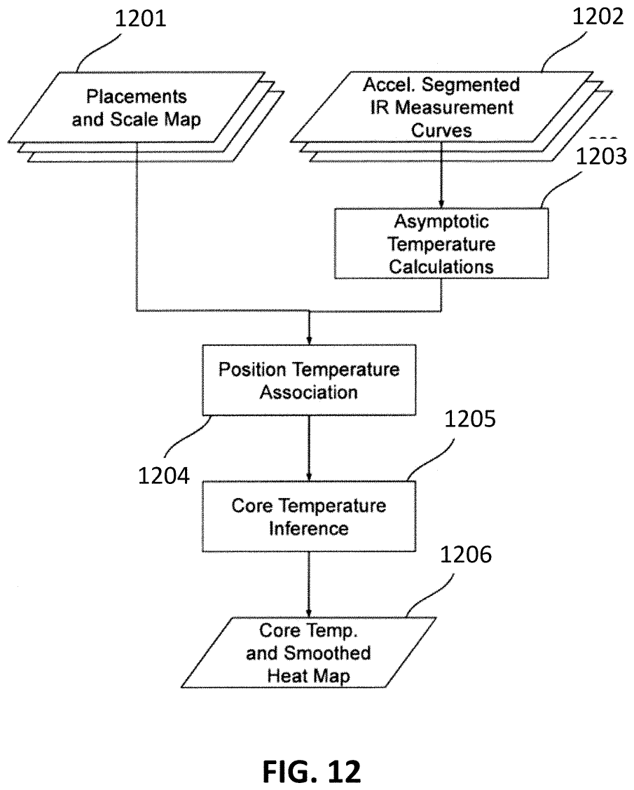

In a further embodiment, the accelerometer 120 is utilized to resolve the occurrence of relocations of the digital stethoscope 100 on a patient. Distinct changes in motion occur during exams which are monitored to automatically segment any data acquired. During digital stethoscope recordings of a patient, users can move from site to site quickly, dwelling at each site for only a few seconds. During the dwell time, the stethoscope is preferably maintained in position with minimal movement. Users can minimize movement to reduce the noise and maintain near optimal positioning. In addition, when relocating or repositioning, users tend to lift, reposition, and place at the site. Adding an accelerometer enables monitoring of accelerations indicative of repositioning (or movement). FIG. 11 illustrates an embodiment of the segmentation of the acceleration signals 1101 that uses a detector bank 1102 that outputs time-varying probability signals for sensing a heartbeat 1103 or relocation 1104, non-target body sounds 1105 (along with any number of other random body sound detectors 1106, e.g., cough that can be analyzed by other processing systems 1108. Additional detectors processing other simultaneously acquired signals (for example, sound) can also be used to generate various other probabilities. The probability of heart sensing (or other desired body sounds) can be compared 1107 with the probability of relocation; whenever the heart signal probability exceeds the relocation probability and is some threshold above zero, a new segment can be defined, if it occurs for some minimum duration 1109. When the relocation probability exceeds the heart sound probability, the segment can be truncated. In this fashion, the original signal 1101 can be separated into one or more segments 1110 for further processing or concise presentation to the user. This automatic segmentation can be enhanced by monitoring the stethoscope noise (or inversely by monitoring for desired sounds) and is useful to place the recording graphically on the body in an application without additional input from the user.

Segmentation and localization of the exam data stream using the accelerometer 120 and/or sound data can enable visuals and interactions. For example, playback of a data stream containing multiple recording sites can be started. In one embodiment, a user can listen to the continuous sound stream while a display highlights the current site source of the currently sounds, as the stethoscope is moved. In an alternative embodiment, multiple recordings can be arranged sequentially or “strung together” and played in exam sequence. In another embodiment can be extended to reordering of exam sequences allowing the recording in any sequence and playback in the preferred sequence by user. Furthermore, segmentation and localization can be further connected with a user interface where sites are selectable. The user can then select a site that corresponds to that portion of the recording and playback the applicable data segment. Adding classification to the segmentation and localization provides the ability to visually label each site as appropriate with detected conditions. For example, the aortic auscultation site can be labeled with valvular disease or high regurgitation while the Erb's point 308 (an auscultation site during a heart examination, located in the third intercostal space close to the sternum) can be labeled with wall motion abnormality. This visual map of health conditions can facilitate user understanding of the exam results.

The accelerometer 120 or other inertial measurement information can be directly integrated, estimating the motion of the stethoscope during recordings. Over short time periods, the estimated movements are typically accurate and can be made more accurate by compensating for drift that might be integrated during the recording when the device is known to be stationary at a site through other cues (for example, the sound data). These estimated changes in position can be used to constrain the optimization of recording positioning.

The accelerometer data can be further processed to determine events occurring in the streams and/or de-interlace source components. For example, the accelerometer data stream can contain overlapping and diverse sources such as motions to new sites, on-site adjustments, chest motion from cough, chest motion from breathing, tremors, body movements, breath holding, valsalva maneuver, etc. An AI model can be applied to the data sequence to find, classify, and separate the signal components due to each event. The AI can produce a signal for each component that is separated. The classifier labels event onsets and enables aggregation and display of exam statistics. The classifier can trigger further AI processing of the sounds, for example, of a cough, which have shown to contain important health information in conditions like pneumonia or COVID-19. The segmentation of accelerometer and/or sound data into each discrete event and/or repeating events can be labeled for training AI models. This pre-demarcation can reduce the time required to create labeled datasets and enables AI- and human-in-the-loop iteration for labeling the dataset where the human identifies examples, edge cases, and errors and the AI can label the bulk of the data from what it has learned so far. The processed events can also be functional or exam procedures, such as breath holding that serves multiple purposes such as modifying the volume of blood returning to the heart while also limiting lung or bronchial sounds. Downstream AI can then be triggered to process only the lower noise version of the heart sounds and/or with added context to the processing of said data.

Algorithms utilizing multisite or multistate data are fed by guidance directing the user to acquire data from each of the sites and/or in each subject state (i.e., orientation, stress level, cycle phase [time of day, time since last drug dosage, menstrual, and more], etc.). These algorithms can greatly expand upon the capabilities of single source algorithms. For example, data from multiple sites can be used to localize the source of signal components and, thus, imply the physical component generating the sounds. Further, multiple states of stress can be used to determine or grade the vitality of individual internal components. Not only does this help with prognostication, but can also reveal issues that can otherwise be hidden. As a non-limiting example, when auscultating a heart at rest it may sound functional, however the added stress of high intensity cardiovascular activity may reveal divergences from a normal sounding heart. As a further example, a user and patient can be guided to use the stethoscope at rest at the mitral auscultation site, then be directed to perform ten minutes of exercise and examine again before the heart begins to relax. Both recordings together contain more information about the state of the heart than either alone.

Embodiments of the subject invention can include multi-state measurements and analysis. As used herein, multi-state refers to more than one measurement of different body positions, activities, stimuli, and/or any other condition in which there is a change in body function or response. For example, recording from the same site (or multiple sites) in an upright position and a reclined position with these changes in state being automatically detected and the data automatically segmented and organized for automatic AI processing and classification using the prior knowledge of the multiple states of the subject. Moreover, the accelerometer can provide a direct measure of the states and any differences therein. The difference between upright and reclined subject measurements can vary between users and the absolute positioning of those states can also vary amongst users. Measuring this accurately captures the spectrum of changes and states possible which is important data for training and decision making driven by AI to maximize results. This is especially true in the context of a self-exam or inexperienced user where the subject cannot strictly adhere to exam guidance (for example, by slouching). Further examples of multi-state include leaning from one side to another, before, during and after surprising or emotional stimuli, or before, during and after exercise.

In a further embodiment, the accelerometer 120 in the digital stethoscope 100 is utilized to measure activity of the subject during at least one of prior to, during, and after a sound recording. This can be achieved with a carry-on person device or as a wearable device. Activity can be a predictor for many health concerns. If an embodiment of the device is carried on or near the patient, for example in a pocket, it can be used to monitor the heart or lungs, and AI can be trained to account for the varied effect of prior activity on heart output. This can increase sensitivity of the AI to abnormalities because of the increased ability to compensate for patient context. In one embodiment, prior activity monitoring can be conducted by, but is not limited to, the stethoscope being carried with the subject. In another embodiment, the carry-on device used to monitor a patient is a wearable device attached to the patient using a strap, adhesive, or some such other means to enable relative station keeping on the patient. The accelerometer can also be embedded, attached, or otherwise arranged on the patient or clothing, a separate activity monitor wirelessly connected to the stethoscope apparatus or an application host mobile device, or in an application host mobile device.

In another embodiment, the accelerometer 120 is utilized to measure motion or vibrations larger or longer in wavelength than the digital stethoscope transducer can measure. One example (depending on the exact digital stethoscope sensor used) is the motion of the chest during inhalation and exhalation. The stethoscope can rise and fall or deflect in and out with each breath of a subject. This pattern of motion can be tracked and used to estimate the relative depths of breaths, the phase of breaths at any moment, and synchronization with other body functions. Many body functions are modulated by nearby pressures (a blood volume effect), for example, there is a breathing effect on heart function. Inhalation creates negative pressure, through movement of the diaphragm and rib cage, drawing more blood volume into the thorax and increasing blood flow to the right side of the heart. In some disease states, breathing is supported by a machine using positive pressure ventilation. Each of these modes has the potential to change pressure in the thorax and alter the volume of blood returning to the right heart from the body. This alteration in blood volume on the right side of the heart is not present in the left side of the heart for several heart beats. The difference in volume can be detected using the PCG. Therefore, tracking breaths can provide valuable context to a heart exam. Moreover, the increased pressure from each breath can act as a multi-state driver for the heart function. Then each heartbeat can be analyzed in the context of varied stress as applied by the diaphragm or support machine. This phenomenon is not limited to the breath—pulse relationship, for example the same framework can be applied to gastrointestinal function.

With the understanding of the pressure and blood volume mediated relationship between the breaths and heartbeats, an estimate can be made as to the volume responsiveness of a patient. That is, if the blood volume preload at the heart increases, the increase in stroke volume (or cardiac output) can be determined. This is possible by processing sounds of blood flow velocity and duration in the heart under varied pressures induced by the diaphragm or support machine. In addition, certain disease states will alter these measurements and can be diagnostic of the disease state. Hypovolemia secondary to dehydration or hemorrhage, for example, could be indicated by alteration of these pressures. Systolic dysfunction is also potentially detectable through monitoring of decreased cardiac output.

In a still further embodiment, an IR sensor 114 of the digital stethoscope 100 is utilized with the sound data and accelerometer data to increase the accuracy of disease detection AI, particularly for conditions known to alter body temperature. The infrared sensor can measure the temperature at each recording site and/or the rate of change of the temperature of a digital stethoscope 100 component that is in contact with the patient. These data can be used to estimate the surface temperature of the patient. When used with the digital stethoscope detected sounds, AI processing accuracy can be improved for pulmonary and cardiac conditions, such as, for example, pneumonia. An increase in body temperature is expected when there is an active infection of the body. The inclusion of temperature with metrics for breathing, coughing, heart output, etc. can build a holistic picture for analysis to determine infection, shock, or other temperature modifying conditions.

Furthermore, combining the temperature readings, obtained with the IR sensor 114, with the estimated position of each reading, can enable more accurate inference of core temperature as external body locations vary with respect to their difference relative to body core temperature. Thus, incorporation of the accelerometer data with the phonocardiographic (PCG) data, as shown, for example, in FIG. 11, can build a multisite temperature map over the body that can be more accurately mapped to a core temperature than a single reading alone or a single site alone. In one embodiment, shown in FIG. 11, the accelerometer segmentation method shown in FIG. 11 is applied to the IR data stream resulting in several IR reading curves 1202, as the membrane 111 of the digital stethoscope 100 has a thermal inertia that is asymptotically overcome by placement of the device in contact with the patient. Knowing the asymptotic nature of the relationship, the terminal temperature can be estimated 1203. The placement and scale information 1201 from accelerometer data or from incorporation of many sensors and processing of those data 1011, as shown in FIG. 10, can be paired 1204 with the calculated thermal reading. These data can be processed using a core temperature AI model 1205. The model can be enhanced using known patient conditions (in shock, sick, BMI, etc.). Using thermodynamic models, the site measurements along with the inferred core temperature can be used to generate a smooth surface temperature map 1206 for presentation to the user.

In another embodiment, the digital stethoscope can have a strain gauge 130 sensor that is utilized on the patient contact side of the digital stethoscope 100. One placement for a strain gauge can be in or on the membrane of the digital stethoscope. Another placement option can be in the bell of the digital stethoscope such that it is protected from patient contact, yet is able to measure small deflections in the structure of the stethoscope when in contact with the patient. The strain gauge can measure one or more pressures applied to the membrane material giving a proxy for the forces applied to an auscultation site. Those forces can potentially indicate a need to displace body fat and, thus, can further indicate the amount of body fat at that location. In a further embodiment, the strain gauge measures the displacement of tissue edema. By way of a non-limiting example, when the digital stethoscope is pressed against the pretibial tissue, the pattern of pressure increase can be used to estimate the level of pitting edema present. Higher levels of edema are noted through smaller rebound pressure of the tissue.

In a further embodiment, a digital stethoscope 100 in combination with the strain gauge 130 can be used to press on the brachial artery until blood flow is obstructed. Then, slowly applying less pressure on the brachial artery, the Korotkoff sounds can be heard. As further pressure is released, the Korotkoff sounds cease. The AI processes the Korotkoff sounds to determine the beginning and end of the sounds and uses the strain gauge values to interpret the systolic and diastolic pressures. This combination can enable a one-device solution, which can eliminate using an external pressure cuff with a standard stethoscope.

A still further embodiment utilizes a combination of the digital stethoscope 100 with a plurality of electrodes 150 on a rim 110 of the digital stethoscope device 100, as shown, for example, in FIG. 2. Using various internal circuits, the electrodes in communication with the patient's skin, can act as sensors for one or more of skin conductance, impedance, and voltage monitors. This enables measurement of electrodermal activity (i.e., changes in skin conductance) as a proxy for sympathetic nervous system activation. In a further embodiment, an electrode is arranged within or on the housing 108 of the digital stethoscope 100, such that when a patient operates the stethoscope their hand is in communication with said electrode in the housing. The patient's hand in contact with the housing electrode forms a circuit through the patient's body to the site of stethoscope placement through one or more rim electrodes. In this fashion, internal circuitry can measure the voltage across the electrodes with or without injection of current, for example as impedance cardiography or electrocardiography, respectively.

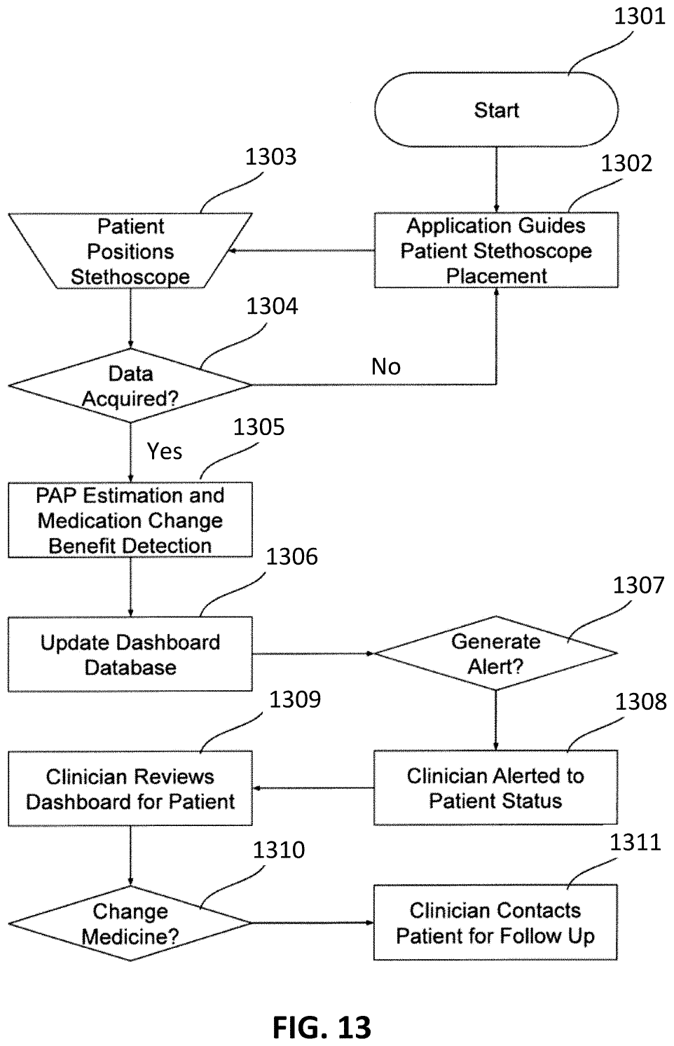

Management of pulmonary artery pressure (PAP) via changes of medication in heart failure (HF) patients has been shown to reduce hospital readmissions. In one embodiment, shown in FIG. 13, a patient can start 1301 an application and can be guided by the application 1302 on a connected device as to an initial placement point 1303 for the digital stethoscope 100 on the body. FIGS. 14 and 15 illustrate a non-limiting example of the self-examination screen 1401 and diagnosis screen 1406 for a mobile application 1302 on a cellular phone 1402 that can be utilized by a user to obtain data with a digital stethoscope 100. The application can further guide the patient if repositioning is required. As shown in FIG. 15, the mobile application can display information about the patient in real-time, such as heart beats per minute (BPM) 1412, respiratory rate 1413, temperature 1414, physical orientation or position 1415, and where the stethoscope is positioned on the body at a given time 1416. There can also be a record button 1408 that can be utilized by the patient to record a session with the digital stethoscope. The mobile application can also show the PCG data 1410 that was obtained, when sufficient data is collected 1304, the data can be sent to a supplemental compute resource 207, such as, for example, a remote computer system for analysis and AI processing. The AI can estimate 1305 the PAP from the sounds and other data collected. Additionally, the AI can directly estimate the need for a change in medication 1305. Results can be added to the patient database 1306. If a change in medication is indicated or abnormal PAP is detected 1307, an alert 1308 can be sent to a clinician. The clinician can review the patient records 1309 and, if necessary, contact 1311 the patient to discuss treatment options 1310. This description of heart failure management is based upon hemodynamic information obtained through the analysis of PCG signals. Estimating blood pressure, pulmonary artery pressure and varying intensity of heart valve murmurs will allow the user to optimize the use of medications that affect blood pressure, heart rate, intravascular fluid balance, inotropy and other characteristics associated with heart failure. These physiologic characteristics are also associated with a wide variety of disease states including acute and chronic kidney disease, renal failure, hypertension and atherosclerotic disease opening the opportunity to manage these disease states on a long-term basis.

Remote health examinations have become commonplace and often require patients to provide information and obtain personal health data utilizing unfamiliar medical equipment and device. The embodiments of the subject invention provide devices and methods that improve the comfort and efficiency of patient handling of the medical equipment or devices utilized for obtaining personal health data. Embodiments of the subject invention provide a digital stethoscope with one or more sensors that can detect sounds and conditions of a patient and can transmit the sounds and information to an AI. Advantageously, the AI can analyze the incoming sound information and determine a direction in which the stethoscope should be maneuvered on the patient body to obtain more accurate information from the patient. When the exam is complete, the AI can analyze the sound and other information received and compare against a cohort of other patients to determine if the sounds and information are indicative of a particular condition. The embodiments described herein are particularly advantageous for detecting and diagnosing heart conditions. When coupled with an application for guiding the patient movements of the digital stethoscope, the patient can more easily and accurately position the digital stethoscope.

The methods and processes of using at least one of the disclosed exam guidance, exam characterization, exam completion determination, attribution interface, and/or attribution-based filtering represent novel inventions from the state of the art. Their combination with a digital stethoscope and/or AI processing for specified use cases represent further inventions disclosed herein. Moreover, many said AI processing apparatus disclosed throughout (i.e., a device processing “heart age” or “lung age”) are novel in their outputs as well as multi-site and/or multi-state inputs. In addition, application of these systems to predict scheduling of interventions represents a new and non-obvious workflow for expert users.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

The examples and embodiments described herein are for illustrative purposes only and that various modifications or changes in light thereof will be suggested to persons skilled in the art and are to be included within the spirit and purview of this application.

The invention has been described herein in considerable detail, in order to comply with the Patent Statutes and to provide those skilled in the art with information needed to apply the novel principles, and to construct and use such specialized components as are required. However, the invention can be carried out by specifically different equipment and devices, and that various modifications, both as to equipment details and operating procedures can be effected without departing from the scope of the invention itself. Further, although the present invention has been described with reference to specific details of certain embodiments thereof and by examples disclosed herein, it is not intended that such details should be regarded as limitations upon the scope of the invention except as and to the extent that they are included in the accompanying claims.

What is claimed is:

1. A system for providing instructions to a user for manipulating a stethoscope to obtain sound from a body of a patient, the system comprising:
a digital stethoscope comprising a vibration transducer that receives and digitizes sound and a transmitter in operable communication with the vibration transducer for transmitting the digitized sound, wherein the digital stethoscope is configured to be placed on the body of the patient in a location to obtain sound;
a user interface device in operable communication with the transmitter of the digital stethoscope, the user interface device comprising a display and being configured to receive the digitized sound transmitted by the transmitter;
a processor in operable communication with the user interface device; and
a machine-readable medium in operable communication with the processor, the machine-readable medium having instructions stored thereon that, when executed by the processor, perform the following steps:
receiving, by the processor, the digitized sound from the user interface device;
analyzing, by the processor, the digitized sound to determine the location of the digital stethoscope relative to the body of the patient;
determining, by the processor, a direction to move the digital stethoscope for relocation on the body of the patient, and
transmitting, by the processor, to the user interface device one or more commands viewable by the user on the display regarding the direction to move the digital stethoscope for relocation on the body of the patient,
wherein, when the stethoscope is relocated, a further sound is received, by the processor, from the body of the patient.

2. The system according to claim 1, wherein the one or more commands for moving the digital stethoscope comprise direct queues that visually indicate the direction to move the stethoscope.

3. The system according to claim 1, wherein the instructions when executed by the processor further perform the following step:
recording, with at least one of the digital stethoscope and the user interface device, at least one of the sound and the further sound from the body of the patient prior to receiving by the processor.

4. The system according to claim 1, wherein the instructions when executed by the processor further perform the following step:
processing, with the user interface device, at least one of the sound and the further sound by one or more of filtering, enhancing, amplifying, segmenting, and dividing into timeframes.

5. The system according to claim 4, wherein the user interface device provides auditory output of the digitized sound to the user.

6. The system according to claim 4, wherein the digitized sound is shown on the display of the user interface device, and wherein the instructions when executed by the processor further perform the following steps:
determining, by the processor, one or more attributes in the digitized sound; and
indicating the position of the one or more attributes on the displayed digitized sound.

7. The system according to claim 1, wherein the digital stethoscope further comprises at least one sensor for obtaining sensor information comprising at least one of biometric, directional, and positional information from the body of the patient, the at least one sensor being in operable communication with the transmitter, and
wherein the digital stethoscope is configured to transmit the sensor information to the user interface device.

8. The system according to claim 7, wherein the at least one sensor comprises a membrane arranged on a bell of the digital stethoscope, an infrared sensor, a strain gauge, a gyroscope, an accelerometer, a compass, and an electrode.

9. The system according to claim 7, wherein the instructions when executed by the processor further perform the following steps:
receiving, by the processor, the sensor information; and
analyzing, by the processor, the sensor information.

10. The system according to claim 9, wherein the instructions when executed by the processor further perform the following step:
displaying the sensor information on the display of the user interface device.

11. The system according to claim 9, wherein the display of the user interface device comprises a homunculus, and
wherein the instructions when executed by the processor further perform the following step:
presenting the commands relative to the homunculus.

12. The system according to claim 10, wherein the instructions when executed by the processor further perform the following steps:
obtaining at least one of a video and image of the body of the patient;
displaying the at least one video and image on the user interface device; and
presenting the commands relative to the at least one video and image.

13. The system according to claim 12, wherein the instructions when executed by the processor further perform the following steps:
utilizing the at least one of the video and image of the body of the patient with a pose estimation model, configured to locate a position for each shoulder of the patent, so as to define a shoulder to shoulder line segment and a bisector line segment in the at least one video and image;
defining a relative coordinate system utilizing the shoulder to shoulder line segment;
utilizing the relative coordinate system to obtain a pose estimation for the patient; and
providing the commands to the user based on the pose estimation.

14. The system according to claim 9, wherein the strain gauge is in operable communication with the membrane arranged on the bell,
wherein, in use, the membrane contacts the body of the patient,
wherein, in use, the strain gauge obtains a measure of pressure applied to the body of the patient by the membrane; and
wherein the instructions when executed by the processor further perform the following step:
analyzing, by the processor, at least one of the digitized sound and the sensor information relative to the pressure measurement.

15. A method for obtaining phonocardiographic (PCG) data from a body of a patient, the method comprising:
providing a digital stethoscope comprising a vibration transducer that receives and digitizes the PCG data and a transmitter operably connected to the vibration transducer for transmitting the PCG data;
receiving from the transmitter the digitized PCG data with a user interface device comprising a display;
transmitting the digitized PCG data with the user interface device to a compute infrastructure resource,
analyzing the PCG data to localize the position of the digital stethoscope on the body of the patient;
calculating a direction to move the digital stethoscope for relocation on the body of the patient;
transmitting to the user interface device one or more instructions viewable on the display regarding the direction for relocating the stethoscope on the body of the patient; and
acquiring additional PCG data from the body of the patient with the relocated digital stethoscope.

16. The method according to claim 15, further comprising the user interface device processing the PCG data by at least one of filtering, enhancing, amplifying, segmenting, and dividing into timeframes prior to transmitting to the compute infrastructure resource.

17. The method according to claim 16, wherein the user interface device provides auditory output of the digitized sound.

18. The method according to claim 15, further comprising,
determining with the compute infrastructure resource one or more attributes in the digitized PCG data,
displaying the digitize PCG data on the display of the user interface device; and
indicating on the display a location of the one or more attributes.

19. The method according to claim 15, wherein the digital stethoscope further comprises at least one sensor for obtaining sensor information comprising at least one of biometric, directional, and positional information from the body of the patient, wherein the at least one sensor is operably connected to the transmitter and the method further comprises transmitting the sensor information to the compute infrastructure resource with the user interface device.

20. The method according to claim 19, wherein the at least one sensor comprises a membrane arranged on a bell of the digital stethoscope, an infrared sensor, a strain gauge, a gyroscope, an accelerometer, a compass, and an electrode.

21. The method according to claim 19, further comprising displaying the sensor information on the display of the user interface device.

22. The method according to claim 19, further comprising:
analyzing the sensor information from the at least one sensor;
combining the analysis of the PCG data with the analysis of the sensor information;
determining a location of the digital stethoscope on the body of the patient;
determining a direction of movement for relocating the stethoscope on the body of the patient;
transmitting instructions to the user interface device viewable by the user for relocation of the digital stethoscope on the body of the patient; and
obtaining a further sound from the body of the patent.

23. The method according to claim 22, wherein the display of the user interface device comprises a homunculus and the method further comprises displaying the instructions relative to the homunculus.

24. The method according to claim 23, further comprising:
obtaining at least one of a video and image of the body of the patient;
displaying the video or image of the body of the patient on the user interface device;
presenting the instructions relative to the video or image.
